(19) United States
(12) Patent Application Publication
(10) Pub. No.: US 2026/0242738 A1
(43) Pub. Date: Aug. 20, 2026

(54) ALK5 INHIBITORS FOR EFFICIENT DERIVATION OF MESENCHYMAL STEM CELLS FROM EMBRYONIC STEM CELLS

(71) Applicant: SCRIPPS HEALTH, San Diego, CA (US)

(72) Inventors: Darryl D. D'LIMA, San Diego, CA (US); Shawn Patrick GROGAN, Mission Viejo, CA (US); Nicholas Edward GLEMBOTSKI, Solana Beach, CA (US)

(21) Appl. No.: 19/130,777

(22) PCT Filed: Nov. 14, 2023
(86) PCT No.: PCT/US2023/079699

§ 371 Date: May 16, 2025

Related U.S. Application Data
(60) Provisional application No. 63/384,289, filed on Nov. 18, 2022.

(51) Int. Cl. C12N 5/0775; C12N 5/00; C12N 5/071

(52) U.S. Cl. CPC C12N 5/0662; C12N 5/0062; C12N 5/0697; C12N 2500/90; C12N 2500/98; C12N 2501/15; C12N 2501/727; C12N 2506/02; C12N 2513/00; C12N 2533/54; C12N 2533/80

(57) ABSTRACT

Provided herein are methods for deriving mesenchymal stem cells from embryonic stem cells. In some cases, the methods provided herein involve the use of ALK5 inhibitors.

### CROSS-REFERENCE

This application claims benefit of U.S. Provisional Application No. 63/384,289 filed Nov. 18, 2022, which is incorporated herein by reference in its entirety.

### BACKGROUND

Successful tissue regeneration requires a clinically viable source of mesenchymal stem cells (MSCs).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE DISCLOSURE

In some aspects, disclosed herein is a method of generating mesenchymal stem cells (MSCs), comprising: culturing a population of pluripotent stem cells in xeno-free conditions; and contacting the population of pluripotent stem cells with an inhibitor of the SMAD2/3 signaling, wherein the population of pluripotent stem cells differentiates into a population of MSCs.

In some embodiments, the method further comprises, after (a), culturing the population of pluripotent stem cells in suspension spinner culture.

In some embodiments, the population of pluripotent stem cells is xeno-free derived ESCs. In some embodiments, the population of pluripotent stem cells comprises HADC 100 cell line.

In some embodiments, the MSCs comprise chondroprogenitor cells.

In some embodiments, the inhibitor of the SMAD2/3 signaling is an ALK-5 inhibitor. In some embodiments, the inhibitor of the SMAD2/3 signaling comprises at least a concentration of 5 μM. In some embodiments, the ALK-5 inhibitor is SB525334. In some embodiments, the ALK-5 inhibitor is SB431542. In some embodiments, the ALK-5 inhibitor is GW788388.

In some embodiments, the population of MSCs is passaged at least two times.

In some embodiments, the population of MSCs are at least 80% positive for CD73, CD90, CD105, or a combination thereof. In some embodiments, the population of MSCs are at least 90% positive for CD73. In some embodiments, the population of MSCs are at least 80% positive for CD90. In some embodiments, the population of MSCs are at least 75% positive for CD105.

In some embodiments, the method further comprises: c) passaging and culturing the population of MSCs to form high-density pellets in a medium; and d) implanting the high-density pellets into ex vivo explants to form a neo-cartilage tissue, a neo-muscular tissue, a neo-meniscus, a neo-tendon, or a neo-bone. In some embodiments, the method comprises culturing the high-density pellets for at least three days before implantation. In some embodiments, the medium is serum-free. In some embodiments, the medium comprises a protein of a TGFβ family. In some embodiments, the TGFβ family comprises TGFβ1, TGFβ2, or TGFβ3.

In some embodiments, the high-density pellets express one or more markers selected from: COL2A1, ACAN, COMP and SOX9. In some embodiments, the high-density pellets express significantly less COL10A1 compared to pellets not derived from xeno-free human pluripotent stem cells. In some embodiments, the high-density pellets express significantly less RUX2A compared to pellets not derived from xeno-free human pluripotent stem cells. In some embodiments, the neo-cartilage tissue, the neo-muscular tissue, the neo-meniscus, the neo-tendon, or the neo-bone expresses GAGs and collagen type II.

In some embodiments, the method further comprises: (c) adding the population of MSCs to a hydrogel construct; and (d) implanting the hydrogel construct into ex vivo explants to form a neo-cartilage tissue, a neo-muscular tissue, a neo-meniscus, a neo-tendon, or a neo-bone. In some embodiments, the hydrogel construct is supplemented with extracellular matrix components. In some embodiments, the extracellular matrix components comprise collagen type II, hyaluronate, chondroitin sulfate, or any combination thereof.

In some embodiments, the ex vivo explants comprise human osteoarthritic cartilage. In some embodiments, the neo-cartilage tissue, the neo-muscular tissue, the neo-meniscus, the neo-tendon, or the neo-bone does not comprise mineralization. In some embodiments, the neo-cartilage tissue, the neo-muscular tissue, the neo-meniscus, the neo-tendon, or the neo-bone comprises mineralization.

In some aspects, disclosed herein is a composition, comprising: a population of pluripotent stem cells in xeno-free culture; and an inhibitor of SMAD2/3 signaling, wherein the inhibitor of SMAD2/3 signaling differentiates the population of pluripotent stem cells to a population of mesenchymal stem cells (MSCs). In some embodiments, the population of pluripotent stem cells comprises a HADC 100 cell line. In some embodiments, the population of pluripotent stem cells comprise chondroprogenitor cells.

In some embodiments, the inhibitor of SMAD2/3 signaling is an ALK-5 inhibitor. In some embodiments, the inhibitor of SMAD2/3 signaling comprises at least a concentration of at least 5 μM. In some embodiments, the ALK-5 inhibitor is SB525334. In some embodiments, the ALK-5 inhibitor is SB431542. In some embodiments, the ALK-5 inhibitor is GW788388.

In some embodiments, the population of MSCs are passaged at least two times. In some embodiments, the population of MSCs are at least 80% positive for CD73, CD90, and CD105, or a combination thereof. In some embodiments, the population of MSCs are at least 90% positive for CD73. In some embodiments, the population of MSCs are at least 80% positive for CD90. In some embodiments, the population of MSCs are at least 75% positive for CD105.

In some embodiments, the population of MSCs are passaged and cultured to form high-density pellets in a medium. In some embodiments, the population of MSCs are added to a hydrogel construct. In some embodiments, the high-density pellets express one or more markers selected from: COL2A1, ACAN, COMP and SOX9. In some embodiments, the high-density pellets express significantly less COL10A1 compared to pellets not derived from xeno-free human pluripotent stem cells. In some embodiments, the high-density pellets express significantly less RUNX2 compared to pellets not derived from xeno-free pluripotent stem cells. In some embodiments, the high-density pellets are implanted into ex vivo explants to a neo-cartilage tissue, a neo-muscular tissue, a neo-meniscus, a neo-tendon, or a neo-bone. In some embodiments, the hydrogel construct is supplemented with extracellular matrix components. In some embodiments, the extracellular matrix components comprise collagen type II, hyaluronate, chondroitin sulfate, or any combination thereof. In some embodiments, the hydrogel construct is implanted into ex vivo explants to form a neo-cartilage tissue, a neo-muscular tissue, a neo-meniscus, a neo-tendon, or a neo-bone.

In some embodiments, the ex vivo explants comprise human osteoarthritic cartilage. In some embodiments, the neo-cartilage tissue, the neo-muscular tissue, the neo-meniscus, the neo-tendon, or the neo-bone does not comprise mineralization. In some embodiments, the neo-cartilage tissue, the neo-muscular tissue, the neo-meniscus, the neo-tendon, or the neo-bone comprises mineralization.

In some aspects, disclosed herein is a population of cells, wherein the population is at least 90% positive for CD73; at least 80% positive for CD90; and at least 75% positive for CD105; wherein the population of cells has a reduced expression of one or more hypertrophic markers.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 depicts a xeno-free approach to generate mesenchymal stem cell (MSC)-like cells from embryonic stem cell (ESC). Panels A and B: ESC on human fibroblast feeders. Panels C and D: ESC on a cell free substrate (CellStart). Panel E: Spinner flask to generate cell clusters over 24 hours. Panel F: Cell clusters following spinner culture ranged in size from 50 μm to 100 μm, with an average size of 75.8±17.7 μm. Panels G and H: After 5-10 days of small molecule exposure in low adherent plates, cell clusters were seeded onto fibronectin coated flasks to allow cell outgrowth for 4 days. Panels I and J: Typical morphology of ES-MSC derived cells by day 21. Panel K: Overview of the differentiation process.

FIG. 2 depicts mesodermal, mesenchymal, and chondrogenic gene expression levels relative to undifferentiated ESC (*P<0.05 compared to undifferentiated ESC). H9 SB43=H9-ES-MSC treated with SB431542; HAD SB43=HADC100-ES-MSC treated with SB431542; HAD SB52=HADC100-ES-MSC treated with SB525334; HAD GW7=HADC100-ES-MSC treated with and GW788388; HAD DMSO=HADC100-ES-MSC treated with DMSO (control).

FIG. 3 depicts flow cytometric evaluations of MSC surface markers for comparison of cells following treatment of H9 ESC with SB431542 or HADC100 ESC with three different ALK5 inhibitors and DMSO. SB43=SB431542; SB52=SB525334; GW7=GW788388 (*P<0.05).

FIG. 4 depicts chondrogenic differentiation of H9 derived ES-MSC. Panels A-H: Safranin O/fast green stained 3-week cultured pellets from four different differentiations. Panel I: Safranin O/fast green stained 3-week cultured pellet. Panel J: Collagen type II immunostained pellet. Panel K: Collagen type I immunostained pellet. Panel L: Isotype control staining. Panels M-R: Safranin O/fast green staining of pellets implanted in ex vivo human OA cartilage and cultured for 3 weeks.

FIG. 5 depicts pellet culture and ex vivo implantation of HADC 100 derived ES-MSC differentiated with different ALK5 inhibitors. Panel A: Histology of pellets derived from DMSO (control), SB431542 (SB43), SB525334 (SB52) or GW788388 (GW7). Pellets were either immunostained for collagen type I, collagen type II or stained with Safranin O/fast green. Panel B: Histology of various pellets implanted into osteoarthritic ex vivo tissues.

FIG. 6 depicts a direct comparison between the relative gene expression profiles of pellet cultured cells derived from either H9-ES-MSC (H9) or HADC 100 ES-MSC (HAD) and differentiation using SB431542 treatment (*P<0.04).

FIG. 7 depicts creation of implantable constructs with fibrin-ECM hydrogels. Panel A: After 3 weeks on a needle array using HAD-MSC (SB43 treatment). Panels B and C: Safranin O staining of 3-week-old cultured fibrin-ECM construct. Panel D: Gene expression profile of several Fibrin-ECM constructs (N=8). Panel E: Safranin O staining of ex vivo OA cartilage explants with either fibrin-ECM or pellets using HAD-MSC derived by three different ALK-5 inhibitor treatments.

FIG. 8 depicts germ layer characterization relative to undifferentiated ESC. Pluripotent (POU5F1, SOX2), Endoderm (SOX17, FOXA1) and Ectoderm (PAX6, NES) genes. * P<0.05 compared to undifferentiated ESC. H9 SB43=H9-ES-MSC treated with SB431542; HAD SB43=HADC100-ES-MSC treated with SB431542; HAD SB52=HADC100-ES-MSC treated with SB525334; HAD GW7=HADC100-ES-MSC treated with and GW788388; HAD DMSO=HADC100-ES-MSC treated with DMSO (control).

FIG. 9 depicts representative flow cytometric evaluations of MSC surface marker expression patterns for H9 (n=5) and HADC100 (n=4) ESC following SB431542 treatment (10 μM, 10 days). Blue histograms=isotype controls; Red histograms=target surface molecule.

FIG. 10 depicts representative Alizarin Red S staining showing the absence of mineralization by ES-MSC in pellet cultures, pellets implanted into ex vivo defects, or ES-MSC implanted in fibrin-ECM in ex vivo defects. Positive control=HAD cells in osteogenic medium for 3 weeks.

FIG. 11 depicts use of microspheroid building block approach to produce bone and cartilage (osteochondral) tissues from ES-MSC derived from ALK-5 inhibitor (SB525334) treatment. Panel A: ES-MSC microspheroids. Panels B and C: Microspheroid building chamber. Panel D: Neo-cartilage tissue derived from ES-MSC. Panels E and F: Positive staining for cartilage glycosaminoglycans (GAGs). Panel G: Positive staining for collagen type II. Panel H: ES-MSC microspheroids pre-differentiated towards cartilage or towards a calcified cartilage phenotype (Osteo MS) both seeded into a chamber. Panel I: Integration of osteochondral (OC) tissue unit with cartilage and osteo MS. Panel J: Safranin O staining of upper and lower tissues after implantation of OC tissue unit into an ex vivo model. Panel K: Collagen type II staining of upper and lower tissues after implantation of OC tissue unit into an ex vivo model. Panel L: von Kossa staining of lower tissue after implantation of OC tissue unit into an ex vivo model. Panel M: Osteopontin staining of lower tissue after implantation of OC tissue unit into an ex vivo model.

FIG. 12 depicts meniscus cartilage-like tissues produced from ES-MSC derived ALK-5 inhibitor (SB525334) treatment that were seeded upon blow spun collagen scaffolds in in vitro and in vivo. Panel A: Safranin O staining after culturing ES-MSC seeded upon a glutaraldehyde crosslinked pneumatospun collagen type I scaffold for 6 weeks. Panels B-E: Safranin O staining profile of meniscus cartilage-like tissue in a nude mouse implanted with ES-MSCs.

FIG. 13 depicts Safranin O, Collagen type I, and Collagen type II staining of meniscus cartilage-like tissues produced from ES-MSC derived ALK-5 inhibitor (SB525334) treatment that were seeded upon blow spun collagen scaffolds with conjugated growth factors (TGFb1, TGFβ3, or PDGFbb) and implanted into ex vivo human meniscus defects.

### DETAILED DESCRIPTION

A variety of approaches have been used to generate mesenchymal stem cell (MSC)-like cells ranging from spontaneous differentiation to methods that specifically target certain pathways via sequential exposure to various molecules and culture conditions to recapitulate embryonic development.

Embryonic stem cells (ESCs) spontaneously differentiate when cultured on standard tissue culture plastic, or on Matrigel or gelatin coatings. Such spontaneous differentiation of ESCs (e.g., H9 ESC) on standard tissue culture plastic has previously been demonstrated, whereby the emergence of MSC-like cells increased with each subsequent passage with the capacity to form cartilage. An alternate approach involves initially directing ESC towards the primitive streak stage using a combination of molecules including Activin A, BMP2, BMP4, FGF2, Wnt3a and CHIR99021 for a few days, followed by the addition of bFGF and Follistatin or the inhibition of BMP receptors with dorsomorphin (DM) to induce paraxial mesoderm differentiation. Subsequent monolayer culture on various substrates led to the emergence of an MSC-like phenotype. This approach recapitulates the complex stages of embryonic development and is a reproducible and well characterized process to derive MSC and chondroprogenitors.

However, one of the major issues limiting clinical translation of ES-MSC is exposure to animal feeder cells or animal serum during culture or cryopreservation, which poses a risk of undesired xenogeneic contamination. Human derived feeder cells, such as fetal fibroblasts, fetal muscle cells, adult dermal fibroblasts, and fallopian tubal epithelial cells have been used to address this issue. Cell-free and xeno-free substrates have also been proposed including naturally derived extracellular matrices such as vitronectin, laminin and fibronectin or synthetic substrates such as, PMVE-alt-MA, APMAAm, PMEDSAH, and PAM₆-co-PSS₂. Serum- and xeno-free reagents are also available for cell expansion and cryopreservation.

For safe clinical use, derivation of ES-MSC may be performed in completely xeno-free environments. Currently, there are several ESC lines that have been successfully derived in xeno-free conditions. Xeno-free HAD-C100, 102, and 106 cell lines have been used for Phase I/II clinical trials for retinal degeneration and amyotrophic lateral sclerosis. In 2018, four more clinical grade xeno-free and feeder-free human ESC cell-lines (HAD-C 103, 104, 105 and 107) were made available.

To date, no clinical trials have been listed on clinicaltrials.gov using ESC-derived cells for cartilage tissue regeneration. Derivation of MSC and chondroprogenitor cells under xeno-free conditions will reduce regulatory hurdles and may facilitate the use of human ESC in clinical trials for cartilage regeneration.

Provided herein are compositions and methods of deriving MSCs from pluripotent stem cells (e.g., human ESCs). Provided herein is a method for rapid and scalable differentiation of ESC to an MSC-like phenotype which led to chondroprogenitors capable of robust neo-cartilage formation in vitro that integrated into ex vivo osteoarthritic tissue. In some cases, the methods provided herein involve the use of inhibitors of SMAD2/3 signaling (e.g., ALK5 inhibitors) to derive MSCs from pluripotent stem cells (e.g., human ESCs).

### Mesenchymal Stem Cells (MSCs)

Mesenchymal stem cells (MSCs) have been widely used as chondroprogenitors for tissue regeneration. In some embodiments, a population of MSCs are derived from pluripotent stem cells (PSCs). In some embodiments, a population of MSCs are derived from embryonic stem cells (ESC). In some embodiments, a population of MSCs are derived from induced pluripotent stem cells (iPSCs). In some embodiments, a population of MSCs are derived from human pluripotent stem cells (PSCs). In some embodiments, a population of MSCs are derived from human embryonic stem cells (ESC). In some embodiments, a population of MSCs are derived from human induced pluripotent stem cells (iPSCs).

In some embodiments, a population of MSCs derived from pluripotent stem cells (e.g., human ESCs) have enhanced proliferative capacity compared to MSCs isolated from tissues, such as skeletal muscle, dental pulp, bone, umbilical cord, placenta, adipose tissue or variant thereof. In some embodiments, MSCs derived from pluripotent stem cells (e.g., human ESCs) have enhanced immunosuppressive properties compared to MSCs isolated from tissues (e.g., skeletal muscle, dental pulp, bone, umbilical cord, placenta, adipose tissue or variant thereof).

In some embodiments, the population of MSCs comprise chondroprogenitor cells. In some embodiments, the population of MSCs expresses higher levels of mesoderm specific markers (e.g., NCAM1, MSC1, NKX2-5) compared to pluripotent stem cells (e.g., human ESCs) without treatment with inhibitor of SMAD 2/3 signaling. In some embodiments, the population of MSCs expresses higher levels of ITGB 1 compared to pluripotent stem cells (e.g., human ESCs) without treatment with inhibitor of SMAD 2/3 signaling. In some embodiments, the population of MSCs expresses at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold more NCAM1, MSC1, NKX2-5, ITGB1 compared to pluripotent stem cells (e.g., human ESCs) without treatment with inhibitor of SMAD 2/3 signaling.

In some embodiments, the population of MSCs is highly enriched for MSC markers. Non-limiting markers of MSCs include CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD73, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD73. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD90. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD105. Percentage of MSCs expressing one or more of the MSC markers disclosed herein can be measured via fluorescence-activated cell sorting (FACS).

### SMAD 2/3 Signaling

In some aspects, disclosed herein are inhibitors of SMAD 2/3 signaling. Inhibition of SMAD 2/3 signaling in pluripotent stem cells (e.g., human embryonic stem cells (ESCs)) can induce mesenchymal stem cell (MSC)-like or MSC phenotype in pluripotent stem cells (e.g., ESCs).

In some embodiments, an inhibitor of SMAD 2/3 signaling is an inhibitor of TGFb1.

In some embodiments, an inhibitor of SMAD 2/3 signaling is a BRD4 inhibitor. Non-limiting examples of BRD4 inhibitors include LLY-507, AZD5153, or variant thereof.

In some embodiments, an inhibitor of SMAD2/3 signaling is a TGFbR-1 (ALK5) inhibitor. In some embodiments, an inhibitor of SMAD2/3 signaling is an ALK5 inhibitor. Non-limiting examples of ALK5 inhibitors include RepSox, SD-208, EW-7197, SB505124, SB431542, SB525334, GW788388, or variant thereof. In some embodiments, an ALK5 inhibitor is SB431542. In some embodiments, an ALK5 inhibitor is SB525334. In some embodiments, an ALK5 inhibitor is GW788388.

In some embodiments, an ALK5 inhibitor blocks the ATP-binding domains of activin receptor-like kinase (ALK) receptors 4, 5, and 7 to inhibit SMAD 2/3 signaling and enhance SMAD 1/5/8/signaling. In some embodiments, an ALK5 inhibitor decreases SMAD 2/3 phosphorylation. In some embodiments, decrease in SMAD 2/3 phosphorylation reduces expression of NANOG, SOX2, OCT4, or combination thereof. In some embodiments, decrease in SMAD 2/3 phosphorylation alters methylation of OCT4, NANOG, or combination thereof. In some embodiments, an inhibitor of SMAD 2/3 signaling (e.g., ALK5 inhibitor) increases BMP4 expression. In some embodiments, increase in BMP4 expression directs pluripotent stem cells (e.g., ESCs) to a mesoderm fate. In some embodiments, an inhibitor of SMAD 2/3 signaling (e.g., ALK5 inhibitor) does not affect BMP4 expression.

### Compositions

In some aspects, disclosed herein is a composition, comprising: a) a population of pluripotent stem cells (e.g., embryonic stem cells (ESCs)); and b) an inhibitor of SMAD 2/3 signaling, wherein the inhibitor of SMAD2/3 signaling differentiates the population of pluripotent stem cells (e.g., ESCs) to a population of mesenchymal stem cells (MSCs).

In some embodiments, the population of pluripotent stem cells is xeno-free derived pluripotent stem cells (e.g., human embryonic stem cells (ESCs)). In some embodiments the population of pluripotent stem cells is xeno-free derived ESCs. In some embodiments, the population of pluripotent stem cells comprises HADC 100 cell line. In some embodiments the population of pluripotent stem cells is not derived from xeno-free pluripotent stem cells (e.g., human ESCs). In some embodiments the population of pluripotent stem cells is not derived from xeno-free ESCs. In some embodiments, the population of pluripotent stem cells comprises H9 ESC cell line.

In some embodiments, the population of pluripotent stem cells (e.g., embryonic stem cells (ESCs)) is in xeno-free conditions. In some embodiments, the population of pluripotent stem cells (e.g., ESCs) is in conditions that is not xeno-free.

In some embodiments, the inhibitor of SMAD 2/3 signaling is an ALK5 inhibitor. In some embodiments, the ALK5 inhibitor is a RepSox, SD-208, EW-7197, SB505124, SB431542, SB525334, GW788388, or variant thereof. In some embodiments, the ALK5 inhibitor is SB431542. In some embodiments, the ALK5 inhibitor is SB525334. In some embodiments, the ALK5 inhibitor is GW788388.

In some embodiments, the population of MSCs comprise chondroprogenitor cells. In some embodiments, the population of MSCs expresses higher levels of mesoderm specific markers (e.g., NCAM1, MSC1, NKX2-5) compared to pluripotent stem cells (e.g., human ESCs) without treatment with inhibitor of SMAD 2/3 signaling. In some embodiments, the population of MSCs expresses higher levels of ITGB1 compared to pluripotent stem cells (e.g., human ESCs) without treatment with inhibitor of SMAD 2/3 signaling. In some embodiments, the population of MSCs expresses at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold more NCAM1, MSC1, NKX2-5, ITGB1 compared to pluripotent stem cells (e.g., human ESCs) without treatment with inhibitor of SMAD 2/3 signaling.

In some embodiments, the population of MSCs is highly enriched for MSC markers. Non-limiting markers of MSCs include CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD73, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD73. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD90. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD105. Percentage of MSCs expressing one or more of the MSC markers disclosed herein can be measured via fluorescence-activated cell sorting (FACS).

### Methods of Generating Mesenchymal Stem Cells (MSCs)

In some aspects, disclosed herein is a method of generating mesenchymal stem cells (MSCs), comprising: a) culturing a population of pluripotent stem cells, and b) contacting the population of pluripotent stem cells with an inhibitor of SMAD2/3 signaling, wherein the population of pluripotent stem cells differentiates into a population of MSCs.

In some embodiments the population of pluripotent stem cells is xeno-free derived pluripotent stem cells (e.g., human embryonic stem cells (ESCs)). In some embodiments the population of pluripotent stem cells is xeno-free derived ESCs. In some embodiments, the population of pluripotent stem cells (e.g., human ESCs) comprises HADC 100, HADC 102, HADC 103, HADC 104, HADC 105, HADC 106, or HADC 107 cell lines. In some embodiments, the population of pluripotent stem cells (e.g., human ESCs) comprises HADC 100 cell line. In some embodiments the population of pluripotent stem cells is not derived from xeno-free pluripotent stem cells (e.g., human ESCs). In some embodiments the population of pluripotent stem cells is not derived from xeno-free ESCs. In some embodiments, the population of pluripotent stem cells comprises H9 ESC cell line.

In some embodiments, the population of pluripotent stem cells (e.g., human ESCs) is cultured in xeno-free conditions. In some embodiments, the population of pluripotent stem cells (e.g., human ESCs) is cultured in conditions that is not xeno-free. In some embodiments, the population of pluripotent stem cells (e.g., human ESCs) is further cultured in suspension spinner culture.

In some embodiments, the inhibitor of SMAD 2/3 signaling is an ALK5 inhibitor. In some embodiments, the ALK5 inhibitor is a RepSox, SD-208, EW-7197, SB505124, SB431542, SB525334, GW788388, or variant thereof. In some embodiments, the ALK5 inhibitor is SB431542. In some embodiments, the ALK5 inhibitor is SB525334. In some embodiments, the ALK5 inhibitor is GW788388. In some embodiments, the inhibitor of SMAD 2/3 signaling comprises a concentration of at least about 0.5 μM, at least about 1 μM, at least about 2 μM, at least about 3 μM, at least about 4 μM, at least about 5 μM, at least about 6 μM, at least about 7 μM, at least about 8 μM, at least about 9 μM, or at least about 10 μM. In some embodiments, the inhibitor of SMAD 2/3 signaling comprises a concentration of at most about 5 μM, at most about 6 μM, at most about 7 μM, at most about 8 μM, at most about 9 μM, at most about 10 μM, at most about 15 μM, or at most about 20 μM.

In some embodiments, the population of MSCs is passaged at least about 1 time, at least about 2 times, at least about 3 times, at least about 4 times, at least about 5 times, at least about 10 times, at least about 15 times, or at least about 20 times. In some embodiments, the population of MSCs is passaged at least about 1 time. In some embodiments, the population of MSCs is passaged at least about 2 times.

In some embodiments, the population of MSCs comprise chondroprogenitor cells. In some embodiments, the population of MSCs expresses higher levels of mesoderm specific markers (e.g., NCAM1, MSC1, NKX2-5) compared to pluripotent stem cells (e.g., human ESCs) without treatment with inhibitor of SMAD 2/3 signaling. In some embodiments, the population of MSCs expresses higher levels of ITGB1 compared to pluripotent stem cells (e.g., human ESCs) without treatment with inhibitor of SMAD 2/3 signaling. In some embodiments, the population of MSCs expresses at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold more NCAM1, MSC1, NKX2-5, ITGB1 compared to pluripotent stem cells (e.g., human ESCs) without treatment with inhibitor of SMAD 2/3 signaling.

In some embodiments, the population of MSCs is highly enriched for MSC markers. Non-limiting markers of MSCs include CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105 or variant thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD44, CD73, CD146, CD166, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% positive for CD73, CD90, CD105, or a combination thereof. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD73. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD90. In some embodiments, the population of MSCs is at least about 50%, at least about 55%, at least about 60%, at least about 65%, at least about 70%, at least about 75%, at least about 80%, at least about 85%, at least about 90%, at least about 95%, at least about 99.5% or 100% for CD105. Percentage of MSCs expressing one or more of the MSC markers disclosed herein can be measured via fluorescence-activated cell sorting (FACS).

### Method of Forming a Neo-Cartilage Tissue, a Neo-Muscular Tissue, a Neo-Meniscus, a Neo-Tendon, or a Neo-Bone

In some aspects, disclosed herein is a method of generating osteoarthritic tissue, comprising: further passaging and culturing the population of mesenchymal stem cells (MSCs) generated from any one of the methods described herein to form high-density pellets in a medium and implanting the high density pellets into ex vivo explants to form an osteoarthritic tissue.

In some embodiments, the population of pluripotent stem cells (e.g., embryonic stem cells (ESCs)) is cultured in xeno-free conditions. In some embodiments, the population of pluripotent stem cells (e.g., ESCs) is cultured in conditions that is not xeno-free.

In some embodiments, the high-density pellets are cultured in medium comprising one or more proteins of the TGFβ family (e.g., TGFβ1, TGFβ2, TGFβ3). In some embodiments, the high-density pellets are cultured in medium comprising TGFβ3. In some embodiments, the high-density pellets are cultured in serum-free medium. In some embodiments, the serum-free medium comprises one or more protein of the TGFβ family (e.g., TGFβ1, TGFβ2, TGFβ3). In some embodiments, the high-density pellets are cultured in serum medium. In some embodiments, the serum medium comprises one or more protein of the TGFβ family (e.g., TGFβ1, TGFβ2, TGFβ3). In some embodiments, the high-density pellets are cultured for at least about 1 day, at least about 2 days, at least about 3 days, at least about 4 days, at least about 5 days or more days before implantation.

In some embodiments, the high-density pellets express one or more chondrogenic genes (e.g., COL2A1, ACAN, COMP, SOX9). In some embodiments, the high-density pellets express one or more hypertrophic markers (e.g., COL10A1) or one or more osteogenic transcription factor (e.g., RUNX2). In some embodiments, the high-density pellets express one or more markers selected from: COL1A1, COL2A1, COL10A1, ACAN, COMP, RUNX2, or SOX9. In some embodiments, the high-density pellets derived from xeno-free pluripotent stem cells (e.g., human ESCs) express significantly less COL1A1, COL2A1, COL10A1, ACAN, COMP, RUNX2, or SOX9 compared to pellets not derived from xeno-free pluripotent stem cells (e.g., human ESCs). In some embodiments, the high-density pellets derived from xeno-free pluripotent stem cells (e.g., human ESCs) express at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 35-fold, at least about 40-fold, at least about 45-fold, or at least about 50-fold less COL1A1, COL2A1, COL10A1, ACAN, COMP, RUNX2, or SOX9 compared to pellets not derived from xeno-free pluripotent stem cells (e.g., human ESCs).

In another aspect, the method of generating osteoarthritic tissue comprises adding the population of MSCs generated from any one of the methods described herein to a hydrogel construct and implanting the hydrogel construct into ex vivo explants to form a neo-cartilage tissue, a neo-muscular tissue, a neo-meniscus, a neo-tendon, or a neo-bone.

In some embodiments, the hydrogel construct is supplemented with extracellular matrix components. Non-limiting examples of extracellular matrix components include collagen type II, hyaluronate, chondroitin sulfate, or any combination thereof.

In some embodiments, the ex vivo explants in any one of the methods disclosed herein is from an animal. In some embodiments, the ex vivo explants in any one of the methods disclosed herein is from a human. In some embodiments, the ex vivo explants in any one of the methods disclosed herein comprise osteoarthritic cartilage, muscular tissue, meniscus, tendon, or bone.

In another aspect, the method of generating osteoarthritic tissue comprises seeding the population of MSCs to a microspheroid building block or chamber to form a neo-cartilage tissue, a neo-muscular tissue, a neo-meniscus, a neo-tendon, or a neo-bone. In some embodiments, the population of MSCs is a population of MSC microspheroids. In some embodiments, the population of MSCs or a population of MSC microspheroids is cultured in the microspheroid building block or chamber for at least about 1 week, at least about 2 weeks, at least about 3 weeks, at least about 4 weeks, at least about 5 weeks, at least about 6 weeks, at least about 7 weeks, at least about 8 weeks, at least about 9 weeks, or at least about 10 weeks. In some embodiments, the neo-cartilage tissue, the neo-muscular tissue, the neo-meniscus, the neo-tendon, or the neo-bone is further implanted into an ex vivo explant.

In another aspect, the method of generating osteoarthritic tissue comprises seeding and culturing the population of MSCs upon a blow spun collagen scaffold, and further implanting the cultured population of MSCs into a subject (e.g., mouse, human) to produce a neo-cartilage tissue, a neo-muscular tissue, a neo-meniscus, a neo-tendon, or a neo-bone. In some embodiments, the blow spun collagen scaffold is glutaraldehyde crosslinked pneumatospun collagen type I scaffold. In some embodiments, the blow spun collagen scaffold is further conjugated with growth factors (e.g., TGFb1, TGFb3, or PDGFbb) or unconjugated (e.g., control). In some embodiments, the populations of MSCs is cultured in the blow spun collagen scaffold for at least about 1 week, at least about 2 weeks, at least about 3 weeks, at least about 4 weeks, at least about 5 weeks, at least about 6 weeks, at least about 7 weeks, at least about 8 weeks, at least about 9 weeks, or at least about 10 weeks.

In some embodiments, the ex vivo explants used in any one of the methods disclosed herein is a osteoarthritic tissue defect. In some embodiments, the ex vivo explants used in any one of the methods disclosed herein is a cartilage defect, bone defect, meniscus defect, muscular tissue defect, or combination thereof. In some embodiments, the ex vivo explant comprises tissue defect (e.g., osteoarthritic, cartilage, bone, meniscus, muscular tissue) of at least about 1 mm, at least about 1.5 mm, at least about 2 mm, at least about 2.5 mm, at least about 3 mm, at least about 3.5 mm, at least about 4 mm, at least about 4.5 mm, at least about 5 mm, at least about 6 mm, at least about 6.5 mm, at least about 7 mm, at least about 7.5 mm, at least about 8 mm, at least about 8.5 mm, at least about 9 mm, at least about 9.5 mm, at least about 10 mm, at least about 15 mm, at least about 20 mm, at least about 25 mm, at least about 30 mm in size.

In some embodiments, the neo-cartilage tissue, the neo-muscular tissue, the neo-meniscus, the neo-tendon, or the neo-bone generated by any one of the methods disclosed herein does not comprise mineralization. In some embodiments, the neo-cartilage tissue, the neo-muscular tissue, the neo-meniscus, the neo-tendon, or the neo-bone generated by any one of the methods disclosed herein does comprise mineralization. In some embodiments, the neo-cartilage tissue, the neo-muscular tissue, the neo-meniscus, the neo-tendon, or the neo-bone generated by any one of the methods disclosed herein does not comprise hypertrophic differentiation. In some embodiments, the neo-cartilage tissue, the neo-muscular tissue, the neo-meniscus, the neo-tendon, or the neo-bone generated by any one of the methods disclosed herein express GAGs. In some embodiments, the neo-cartilage tissue, the neo-muscular tissue, the neo-meniscus, the neo-tendon, or the neo-bone generated by any one of the methods disclosed herein is positive for Safranin O. In some embodiments, the neo-cartilage tissue, the neo-muscular tissue, the neo-meniscus, the neo-tendon, or the neo-bone generated by any one of the methods disclosed herein express collagen type I or collagen type II.

### Method of Treatment

In some aspects, a population of mesenchymal stem cells (MSCs) derived from any one of the methods disclosed herein is used for methods of regenerating osteoarthritic tissues (e.g., cartilage, meniscus, bone, muscular tissue). In some embodiments, the population of MSCs is placed directly into an area of defect (e.g., bone defect, cartilage defect, meniscus defect, muscular tissue defect). In some embodiments, the population of MSCs derived with the method disclosed herein is further cultured to form high-density pellets before being placed into an area of defect (e.g., bone defect, cartilage defect, meniscus defect, muscular tissue defect). In some embodiments, the population of MSCs derived with the method disclosed herein is added into a hydrogel construct before being placed into an area of defect (e.g., bone defect, cartilage defect, meniscus defect, muscular tissue defect). In some embodiments, the population of MSCs derived with the method disclosed herein is cultured in a microspheroid building block before being placed into an area of defect (e.g., bone defect, cartilage defect, meniscus defect, muscular tissue defect). In some embodiments, the population of MSCs derived with the method disclosed herein is cultured in a blow spun collagen scaffold before being placed into an area of defect (e.g., bone defect, cartilage defect, meniscus defect, muscular tissue defect).

In some embodiments, the population of MSCs derived from any one of the methods disclosed herein is integrated into a biomaterial prior to being placed or transplanted into an area of defect (e.g., (e.g., bone defect, cartilage defect, meniscus defect, muscular tissue defect). Non-limiting examples of biomaterials include collagen, polyglycolic acid (PGA), polylactic acid, alginates (for example, the calcium salt), polyethylene oxide, fibrin adhesive, polylactic acid-polyglycolic acid copolymer, proteoglycans, glycosaminoglycans, human dermis, or a combination thereof. In some embodiments, proteoglycans and glycosaminoglycans are sulfated. In some embodiments, the biomaterial is a membrane such as sheet, a porous body such as sponges, a mesh such as a knit, a textile, a non-woven fabric, cotton, and the like. In some embodiments, the biomaterial is porous material.

In some aspects, a population of MSCs derived from any one of the methods disclosed herein is used for methods of repairing or treating an osteoarthritic defect or cartilage-related disorder. In some embodiments, the population of MSCs derived from any one of the methods disclosed herein is administered to the site of the osteoarthritic defect or injury in a subject in need thereof. Non-limiting examples of cartilage-related disorder include arthritis, osteoarthritis, articular cartilage trauma, meniscus injury, a chonodrogenesis disorder, arthritis, chondropathy, chondrosarcoma, chondromalacia, polychondritis, relapsing polychondritis, slipped epiphysis, osteochondritis dissecans, chondrodysplasia, costochondritis, osteochondroma, spondylosis, osteochondroses, Tietze syndrome, dermochondrocorneal dystrophy of Francois, epiphyseal dysplasia, carpotarsal osteochondromatosis, achondropasia, chondrocalcinosis, genochondromatosis, chondroma, achondrogenesis, echondromata, hyprochondroplasia, Keutel syndrome, or variant thereof.

### EXAMPLES

The following is a description of various non-limiting examples of methods and materials used in the studies, and are put forth so as to provide those of ordinary skill in the art with a complete disclosure and description of how to make and use the present disclosure, and are not intended to limit the scope of the disclosure nor are they intended to represent that the experiments below were performed and are all of the experiments that may be performed. Efforts have been made to ensure accuracy with respect to numbers used (e.g., amounts, percentages, etc.), but some experimental errors and deviations should be accounted for.

### Example 1: Methods and Materials

### Cell Sources

Two ESC cell lines (H9 and HADC 100) were studied. The H9 ESC cell line was derived using irradiated mouse embryonic fibroblast feeder layers and cultured in fetal bovine serum, and has been extensively studied. In vitro and ex vivo chondrogenic performance was previously characterized. This cell line is listed in the NIH registry as NIH-line WA 09 and was supplied by WiCell (Madison, WI) as the H9 line. The HADC 100 ESC was derived by the Hadassah Medical Center as previously reported by Tannenbaum et al. Briefly, the HADC 100 ESC line was developed in animal-free and GMP-compliant culture system under cleanroom conditions. Donor eligibility was screened, to follow regulatory guidelines. Each procedure from donor tissue and embryo handling, derivation of hESC, culturing, cryopreservation, characterization, and banking was monitored to satisfy quality practices for use in transplantation therapy. Human NUFF1 foreskin feeder cells were obtained from MTI-GlobalStem (Gaithersburg, MD).

### ESC Expansion

Thawed stocks of H9 ESC were seeded at a density of 1-1.5×10⁵ cells/cm² on CellStart (ThermoFisher, Carlsbad, CA) coated plates in StemPro hESC SFM medium (ThermoFisher) with 8 ng/ml bFGF (ThermoFisher) and 10 μM Y-27632 (EMD Millipore), with daily medium changes. H9 ESC were passaged by dissociation with Accutase® (Innovative Cell Technologies, Inc. San Diego). Prior to cultivation of HADC 100 ESC on CellStart, the cells were defrosted and cultured on human foreskin feeders as previously described. To expand HADC 100 ESC, a feeder cell layer was established by plating mitotically inactivated NUFF1 cells (NUFF1-MMC) onto gelatin (Stem Cell Technologies, Cambridge, MA) coated plates at a concentration of 30,000 cells/cm². Prior to seeding HADC 100 ESC cells, the feeder medium was changed to Nutristem hPSC XF (Biological Industries). Thawed stocks of ESC cells were seeded onto the NUFF1-MMC cells at a density between 10,000-13,000 cells/cm², with medium changes daily. HADC 100 cells were passaged using TrypLE Select for 6 minutes and then seeded onto previously prepared NUFF1-MMC seeded plates (24 hours prior). For feeder-free cultures, HADC 100 ESC were detached using TrypLE Select for 6 minutes and seeded on pre-coated CellStart 6-well plates in StemPro hESC SFM medium (ThermoFisher). For feeder-free expansion, the ESCs were detached with Accutase® and seeded upon the CellStart substrate following the manufacturer's directions (ThermoFisher Scientific, Carlsbad, CA) and using StemPro hESC SFM medium (ThermoFisher).

### ESC to MSC Differentiation

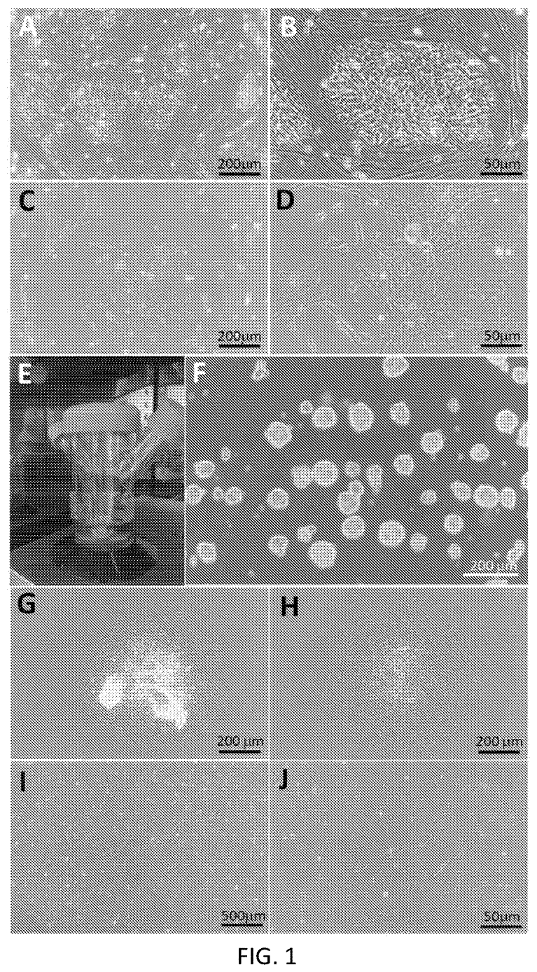

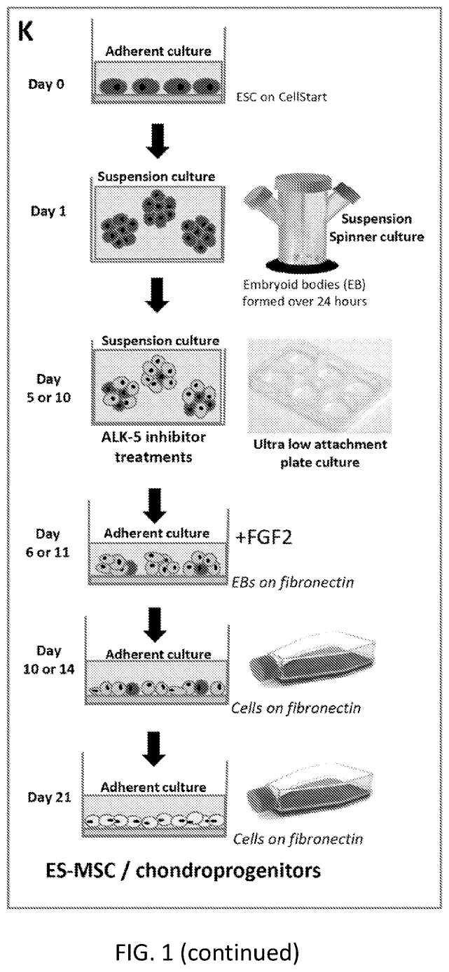

An overview of the differentiation process is provided in FIG. 1, panel K. To create cell clusters or embryoid bodies of uniform size, ESCs were removed from CellStart culture using Accutase® for 2-5 minutes until single cells were observed. Cells (2.5-3×10⁵ cells per ml) were suspended in StemPro human ESC serum free medium (ThermoFisher) with 10 μM Y-27632 (EMD Chemicals, Gibbstown, NJ) and 40 ng/ml FGF (Life Technologies, Carlsbad, CA) in a 125 ml spinner culture flask (Corning Inc., Corning, NY) on a magnetic stirrer (Cimarec™ Biosystem Slow-Speed Stirrer, ThermoFisher) at 70 rpm for 24 hours at 37° C. with 5% CO₂ to form small cell clusters (minimum total cells was 7.5×10⁶ in 30 ml). After 24 hours, the cell clusters were suspended at 2.5-3×10⁵ cells per ml in ultra-low adherent 6 well plates (Corning) in StemPro-34 serum free medium (ThermoFisher) supplemented with 2 mM L-glutamine (ThermoFisher Scientific), 50 μg/ml Ascorbic acid (Stemcell Technologies, Vancouver, Canada) and 1.8 μl/ml ITS-G (100× Insulin-Transferrin-Selenium; ThermoFisher); termed as “complete SP-34 medium”. For ESC to MSC differentiation, the complete SP-34 medium was supplemented with one of three ALK-5 inhibitors: SB431542 (10 μM; Selleckchem, Houston, TX), or SB525334 (10 μM; Selleckchem), or GW788388 (5 μM; Selleckchem) or in 0.01% DMSO (Controls; ThermoFisher Scientific) for 5 or 10 days.

The medium was changed every 2 days by removing cell clusters into 15 ml sterile tubes and permitted to gravity settle for 10 minutes at room temperature. Most of the media was removed using a pipette to leave approximately 300-500 μl of medium. Freshly made medium (with the appropriate ALK5 inhibitor or DMSO) was replaced at the same volumes and well number into new ultra-low adherent plates.

After treatment with the appropriate ALK5 inhibitor, the cell clusters were removed by gravity settling and suspended in complete SP-34 medium supplemented with bFGF 20 ng/ml (without any ALK-5 inhibitor or DMSO) and transferred onto fibronectin (FN) coated (10 μg/ml; Fibronectin from human plasma, Sigma-Aldrich, St. Louis, MO) T75 cm² flasks (passage zero (P0)). The emerging cells and remaining cell clusters were exposed to Accutase® for 5-10 minutes, gently triturated and counted. The cells were seeded at 4.5×10⁶ per cm² in T75 FN coated flask (P1) and medium changed every 3-4 days until 80-90% confluence. At this point, the cells were detached using Accutase® for continued culture, screened for surface marker phenotype characterization (flow cytometry), placed into 3D cultures to assess chondrogenic differentiation capacity, or preserved in liquid nitrogen.

### Pellet Cultures

Monolayer-expanded ES-MSC (passage 2-5) were detached using Accutase® and 0.5×10⁶ cells in 0.5 ml of chondrogenic medium were centrifuged at 900 rpm (~770 G) for 5 minutes to form high-density pellets. At least 5-6 pellets, for each ES-MSC condition, were cultured in serum-free chondrogenic medium consisting of Dulbecco's Modified Eagle Medium (Mediatech Inc., Manassas, VA), 1×ITS supplement (Sigma-Aldrich, St. Louis, MO), 100 nM Dexamethasone (Sigma), 1.25 mg/ml human serum albumin (Bayer, Leverkusen, Germany), 100 μM ascorbic acid 2-phosphate (Sigma), 1% penicillin/streptomycin/gentamycin (PSG, Gibco, Carlsbad, CA), and 10 ng/ml TGFβ3 (PeproTech, Rocky Hill, NJ). Pellets for ex vivo implantation were cultured for 3 days before implantation; pellets for histologic or gene expression analysis were maintained in culture for 3 weeks with media changes every 3 to 4 days.

### ESC Hydrogel Constructs

Monolayer cultured ES-MSC were suspended in a hydrogel wafer composed of collagen II (Innovative Research Inc. Novi, MI), hyaluronate (Supartz FX sodium hyaluronate, Seikagaku Corp. Tokyo, Japan), chondroitin sulfate (Sigma-Aldrich) and fibrin (Baxter Healthcare, Deerfield, IL) at a density of 20×10⁶ cells per ml. The fibrinogen and thrombin components were prepared separately. To each fibrinogen component, 2 mg/ml sodium hyaluronate, 2 mg/ml chondroitin sulfate and 6 mg/ml collagen II was added (2× concentration of final). Cells were added to the thrombin component at a density of 40×10⁶ cells per ml. Thrombin and fibrinogen components in individual syringes were attached to a Baxter DUO mixing syringe and extruded to form the coagulated constructs. The constructs were either directly implanted into ex vivo cartilage tissue or coagulated in 6 well tissue culture plates for 4 to 8 minutes before 4-6 ml of chondrogenic medium was added. To prevent shrinkage of the constructs during culture, the gels were held on AdminPatch microneedle arrays (AdminMed, Sunnyvale, CA) for the duration of their culture.

### Ex Vivo Human Osteoarthritic Cartilage Defect Repair

Osteochondral tissues were obtained from patients ranging between 60 to 70 years of age undergoing total knee arthroplasty within 4-6 hours of surgery (approved by Scripps Institutional Review Board) as previous described. Cartilage disks (6 mm in diameter and ~2-3 mm thick) were harvested from the osteochondral specimens using a sterile dermal punch. The cartilage tissue was cultured in medium consisting of DMEM (Mediatech Inc., Manassas, VA) supplemented with 10% calf serum (Omega Scientific Inc., Tarzana, CA) and 1% Penicillin-Streptomycin-Gentamycin (Life Technologies, Carlsbad, CA). Cartilage disks were maintained in 6-well plates (2-4 per well) in 8 ml medium for 72 hours before creation of the surgical defect. A flame-sterilized stainless-steel burr was used create defects approximately 2×2 mm wide and 0.5 to 1 mm deep. These defects were filled with pellets or hydrogel constructs trimmed to size using 2 mm dermal biopsy punches.

### Gene Expression Profiling

Total RNA was extracted from using the RNeasy kit as recommended by the manufacturer (QIAGEN, Valencia, CA). The High-Capacity cDNA Reverse Transcription Kit (Applied Biosystems, Foster City, California) kit was used to make cDNA and the following verified primer/probe assays were purchased from Applied Biosystems. Expression of the following genes was quantified POU5F1/OCT4 and SOX2 (for pluripotency); FOXA1 and SOX17 (for endoderm); NES and PAX6 (for ectoderm); NKX2-5, MSX1, NCAM1, and SOX9 (for mesoderm); ITGB1/CD29 (for MSC phenotype); and COL1A1, COL2A1, COL10A1, COMP, ACAN, and SOX9 and RUNX2 (for chondrogenesis). Gene expression was normalized to GAPDH as previously reported. Undifferentiated ESC served as a baseline for relative changes in gene expression in differentiated ES-MSC cells. Differentiated ES-MSC cells in monolayer served as baseline for changes in gene expression in pellets or hydrogel constructs.

### Flow Cytometry

Cells were detached using Accutase®, washed and suspended in FACS buffer at a concentration of 0.2×10⁶ cells per 100 μl. A panel of CD molecules were used, each with the following fluorochromes: CD34-FITC, CD45-BV510, CD73-APC (BioLegend, San Diego, California) CD90-PE-Cy7, and CD105-BV650 (BD Biosciences, Franklin Lakes, New Jersey). Matching isotype control IgGs with the same fluorochrome conjugates were used for background gating. For compensation of spectral overlap, unstained cells, and individual tubes with each fluorochrome were incubated with Dynabeads to distinguish between positive and negative fluorochrome signals. Data was acquired with the Novocyte flow cytometer (ACEA Biosciences Inc. San Diego, CA) and analyzed using FlowJo software (version 10, FlowJo, LLC, Ashland, OR) to determine percentage positive signal for each molecule relative to isotype background non-specific signal controls.

### Histology and Immunohistochemistry

Cell pellets and explants were fixed in Z-Fix (3.7% formaldehyde, Anatech Ltd., Battle Creek, MI), processed for embedding in paraffin, and cut into 4 μm-thick sections. Sections were stained with Safranin-O and Fast Green to visualize glycosaminoglycan distribution in the tissues. Alzarin Red S staining was used to detect the presence of mineralized matrix.

For assessment of collagen types I and II, sections were pretreated with pepsin (Digest-All 3, Thermo Fisher Scientific) for 9 minutes at 37° C. in a humid chamber before incubation at 4° C. for 12 to 16 hours with the following primary antibodies: rabbit anti-human collagen type I antibody (Ab 34710, Abcam, Cambridge, MA) 1 μg/ml; or mouse anti-human collagen type II (II-II6B3, Hybridoma Bank, University of Iowa) 2 μg/ml. For color development, the ImmPRESS secondary DAB (brown) or AP (red) kits (Vector Laboratories, Burlingame, CA) was used. Isotype controls were used to monitor non-specific staining.

### Statistical Analysis

Differences in flow cytometry surface markers between H9 and HAD cells were tested for significance using student's t-test. Comparisons between individual surface markers for the different ALK-5 inhibitors and DMSO were tested using a single factor ANOVA and post-hoc Bonferroni corrected t-tests. Significant changes in gene expression between groups were analyzed using the online BootstRatio application, where p-values of less than 0.05 were considered significant.

### Example 2: Embryonic Stem Cell (ESC) Derived Mesenchymal Stem Cell (MSC)

### Xeno-Free Culture Expansion of ESC in Suspension Spinner-Culture

HADC100 ESC cells, defrosted and seeded initially upon mitotically inactivated human NUFF1 foreskin feeder cells (method further detailed in Example 1), rapidly grew in distinctive clusters (FIG. 1, panels A and B). H9 and HADC100 ESC, seeded upon a cell-free substrate of CellStart, acquired a fibroblastic morphology (FIG. 1, panels C and D). At approximately 80-90% confluence, the H9 or HADC100 ESC were detached using Accutase® to achieve a near single cell suspension and transferred to spinner culture flasks (FIG. 1, panel E) for 24 hours to produce cell clusters (FIG. 1, panel F). Cell clusters were exposed to ALK-5 inhibitors for 5-10 days in ultra-low adherent plates (5 days for SB525334, and GW788388, and 10 days for SB431542). Cell clusters were then plated on human fibronectin, without the presence of ALK-5 inhibitor and in the presence of FGF2; and cellular outgrowth was evident by 12-24 hours (FIG. 1, panels G and H). After 4 days, the cells were detached and re-plated onto new fibronectin-coated flasks until confluence. Representative morphology of expanded embryonic stem cell (ES)-MSC is shown in FIG. 1, panels I and J. No differences in cell morphology were observed among the different ALK-5 inhibitor treatments. An overview of the differentiation process is outlined in FIG. 1, panel K.

### ALK-5 Inhibition Induces Rapid Differentiation to an MSC-Like Phenotype

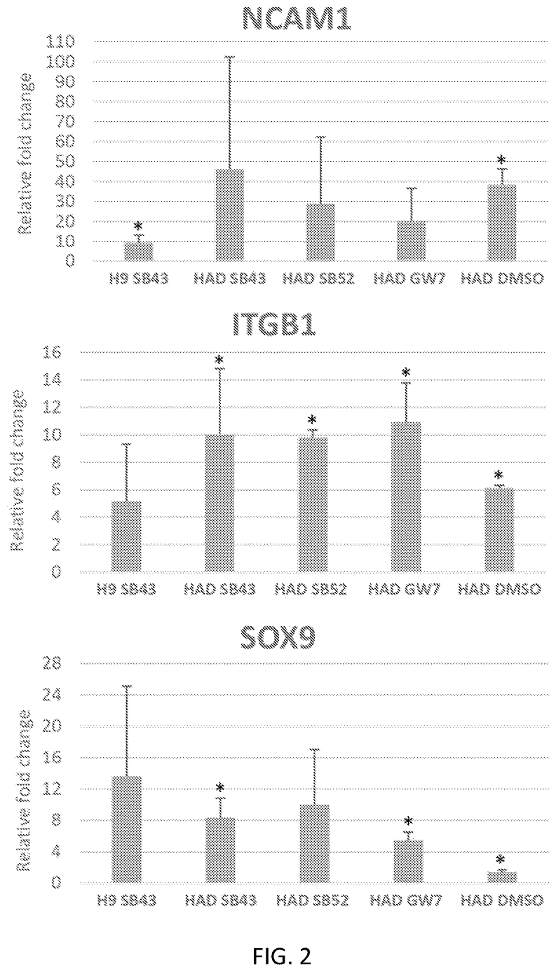

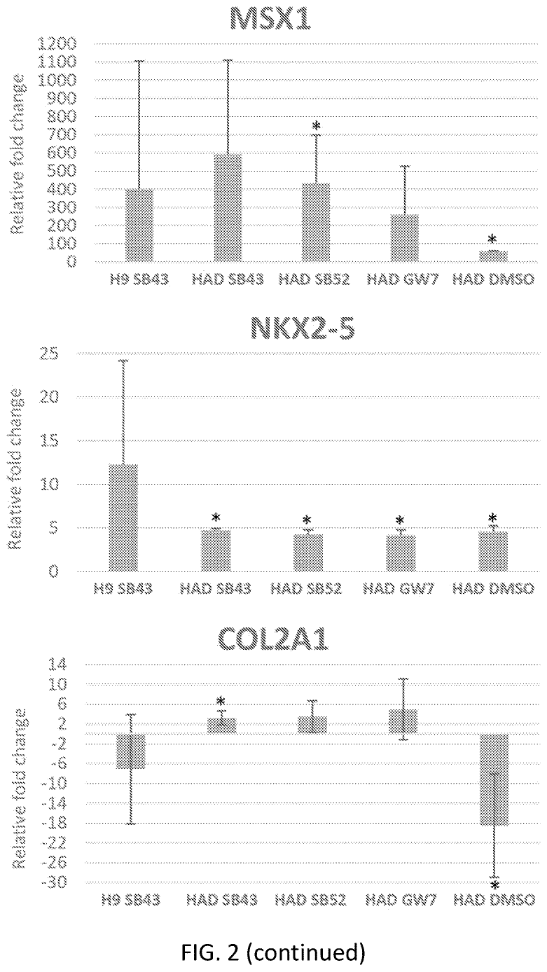

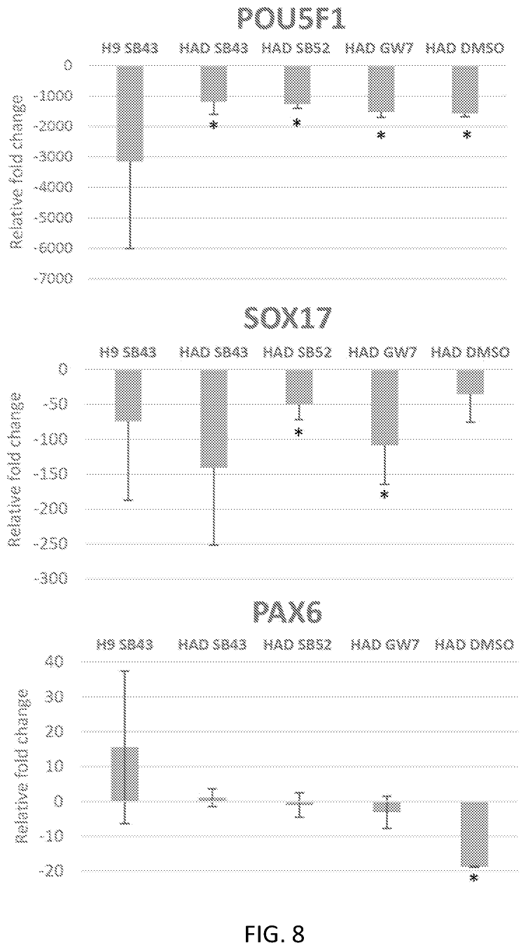

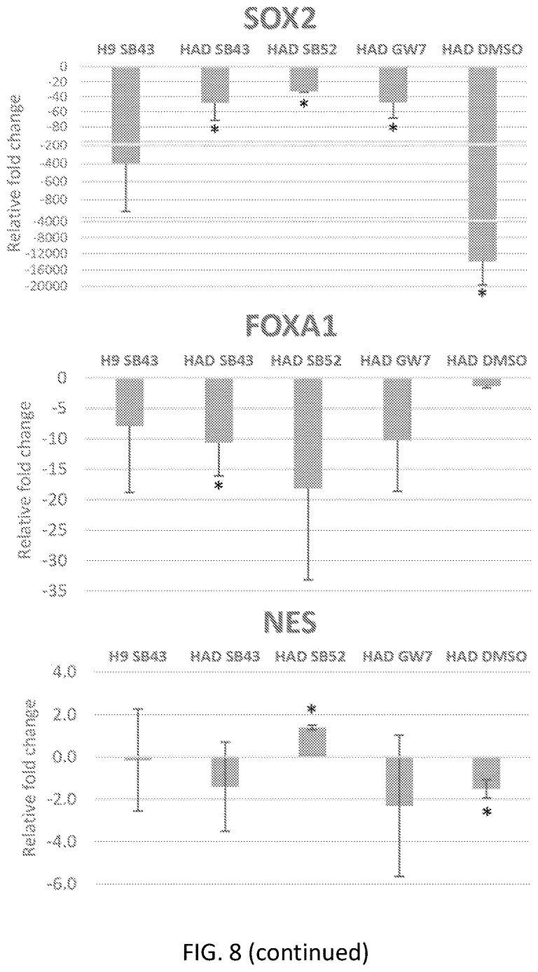

HADC 100 ESC, subjected to one of multiple ALK5 inhibitors (SB431542, SB525334, or GW788388) were compared to H9 ES-MSC (subjected to SB431542) and a DMSO control. Detailed method is outlined in Example 1. After differentiation of H9 ESC into ES-MSC the pluripotent markers POU5F1 (OCT4) and SOX2 were significantly down regulated in the ES-MSC (FIG. 8). The expression of endoderm markers (SOX17, FOXA1, PAX6) remained low in ESC and ES-MSC and ectoderm marker (NES) expression was unchanged by treatment (FIG. 8). On the other hand, mesoderm specific markers (NCAM1, MSX1, NKX2-5) and MSC marker (ITGB1) were significantly upregulated. Increased expression of SOX9 and COL2A1 indicated chondroprogenitor phenotype (FIG. 2). Treatment of the HADC100 ESC with DMSO (control) resulted in spontaneous differentiation towards a mesoderm phenotype (FIG. 2), without specifically inducing a chondroprogenitor phenotype (COL2A1).

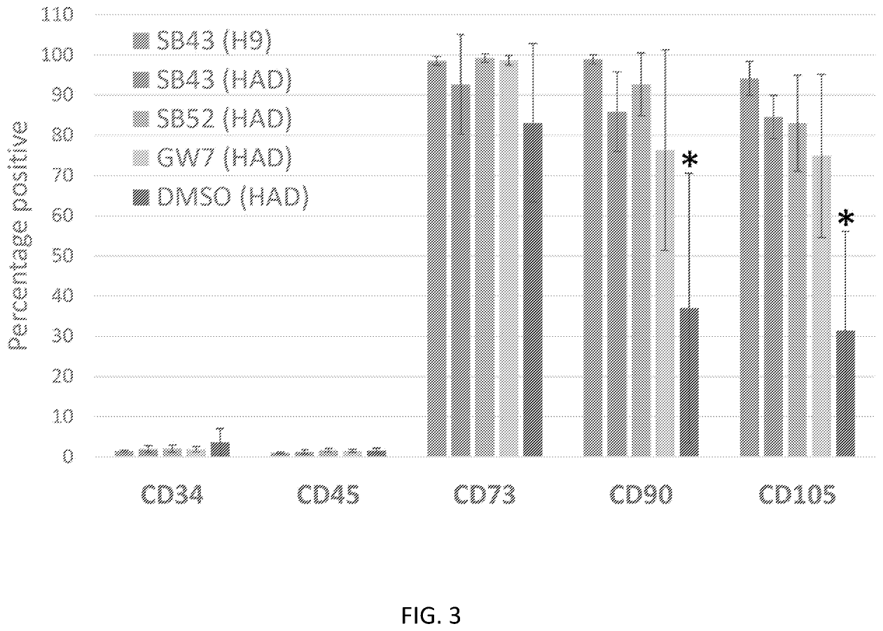

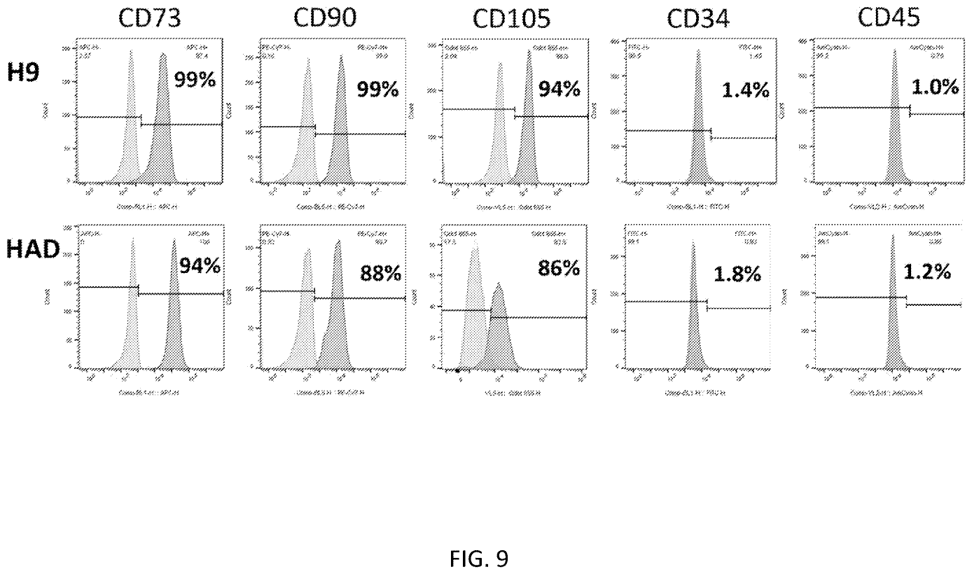

H9 ESC (N=5) and HADC 100 ESC (N=4) were differentiated to ES-MSC using SB431542 (10 μM) for 10 days for a direct comparison between the two cell-lines. A surface marker profile resembling an MSC phenotype was observed (positive for CD73, CD90 and CD105; and negative for hematopoietic markers CD34 and CD45 (FIG. 3 and FIG. 9). H9 ES-MSC displayed significantly (p<0.04) higher percentage levels of CD90 (99±1%) and CD105 (94±4%) compared to the HADC 100 ES-MSC (CD90: 88±10% and CD105: 86±7%). No significant difference for CD73 (H9: 99±1% and HAD: 94±12%) was observed between these two ES-MSC derived cells (FIG. 3). Co-expression analysis revealed that H9-ES-MSC were 92±9% triple positive for CD73, CD90 and CD105; HADC 100 ES-MSC were 88±8% triple positive (not significantly different).

Alternative ALK5 inhibitors SB525334 (10 μM) or GW788388 (5 μM) were compared to SB431542 for derivation of ES-MSC from xeno-free HADC 100 ESC (FIG. 3 and Table 1). The concentrations and duration of exposure were based on initial evaluations. CD73 was highly expressed by all cell treatments (SB431542: 93.6±11.8%; SB525334: 99.2=1.0%; GW788388: 98.9±1.1%) including DMSO controls (83.1±19.7%), with no significant differences between treatments (FIG. 3). Significantly higher expression of CD90 and CD105 was seen in the SB431542 (CD90: 87.6±10.4; CD105: 86.1±6.5), SB525334 (CD90: 93.9±7.6; CD105: 85.4±12.2) and GW788388 (CD90: 80.9±23.9; CD105: 79.6±20.5) treatments compared to the DMSO control (CD90: 37.1±33.5; CD105: 31.4±24.7) (P<0.05; FIG. 3). There were no significant differences for these surface molecules among the three ALK5 inhibitor treatments tested. As stated earlier, SB431542 treated HAD-ESMSC were observed to be 88±8% triple positive, while SB525334 treated ES-MSC were 87±8% triple positive; GW788388 treated cells were 78±13%; and DMSO controls 44±31% triple positive (for CD73, CD90 and CD105).

TABLE 1
Overview of surface marker expression levels for HADC100
ESC exposed to different ALK-5 inhibitors (N = 4).

| Treatment | CD34 | CD45 | CD73 | CD90 | CD105 |
|---|---|---|---|---|---|
| SB43 | 1.8 ± 0.9 | 1.2 ± 0.5 | 93.6 ± 11.8 | 87.6 ± 10.4 | 86.1 ± 6.5 |
| SB52 | 1.9 ± 0.9 | 1.5 ± 0.5 | 99.2 ± 1.0 | 93.9 ± 7.6 | 85.4 ± 12.2 |
| GW7 | 1.8 ± 0.6 | 1.4 ± 0.4 | 98.9 ± 1.1 | 80.9 ± 23.9 | 79.6 ± 20.5 |
| DMSO | 3.6 ± 3.5 | 1.6 ± 0.6 | 83.1 ± 19.7 | 37.1 ± 33.5* | 31.4 ± 24.7* |

*Significantly less (P < 0.05) CD90 and CD105 expression in DMSO controls. Error bars = standard deviation.

Cell Pellets Synthesize Cartilaginous Matrix and Integrate into Ex Vivo Osteoarthritic Cartilage

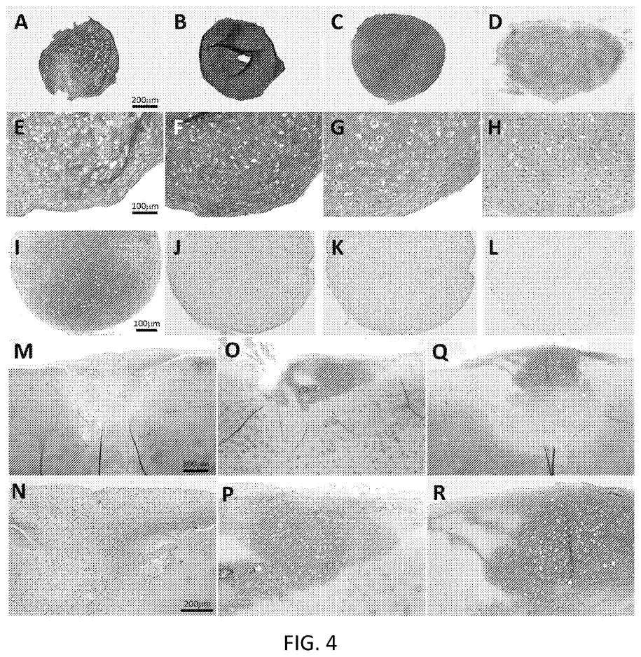

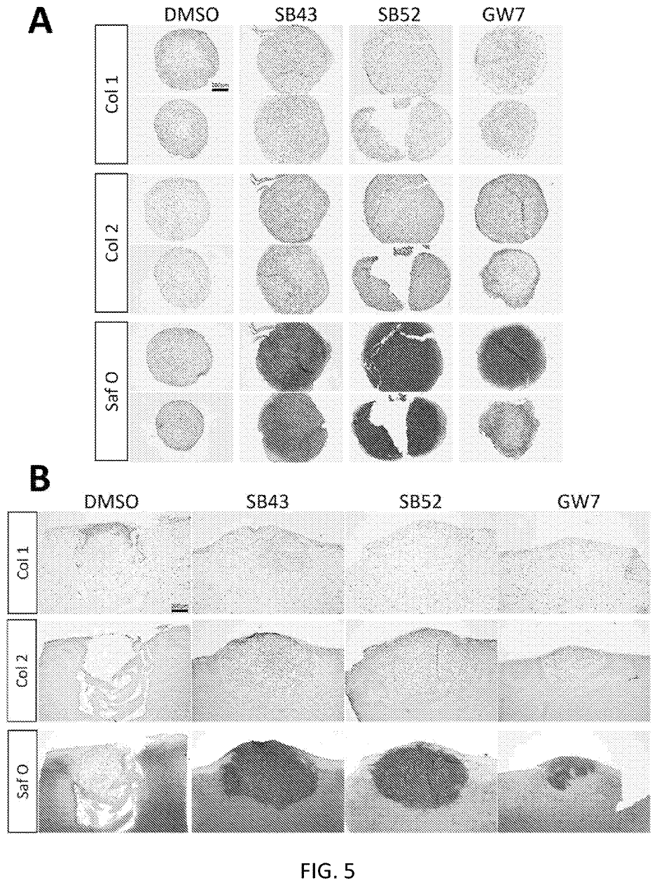

Chondrogenesis in high density pellets of ES-MSC (H9 after SB431542 treatment and HADC 100 after SB431542, SB525334 or GW788388 treatment) was assessed after 3 weeks (FIG. 4 and FIG. 5). Detailed method is outlined in Example 1. Both cell lines (after SB431542 treatment) consistently produced cartilage like-tissue with extensive ECM staining strongly with Safranin-O and evidence of collagen type II, and low collagen type I deposition. HAD-MSC generated with exposure to 2 alternative ALK5 inhibitors (SB525334 and GW788388) also produced cartilage-like tissue. SB525334-treated cells showed the most consistent staining for GAGs and collagen type II, and lower collagen type I signal compared to SB431542 and GW788388. Control (DMSO) treated cells displayed no positive signal for GAGs or collagen type II (FIG. 5, panel A).

The ES-MSC incubated as pellet cultures was also tested and implanted in ex vivo explants of human osteoarthritic cartilage as previously described. Both H9 and HADC 100 ES-MSC pellets generated robust cartilage-like tissue (rich in GAGs and collagen type II) that integrated well with the diseased host tissue (FIG. 4, panels M-R; and FIG. 5, panel B).

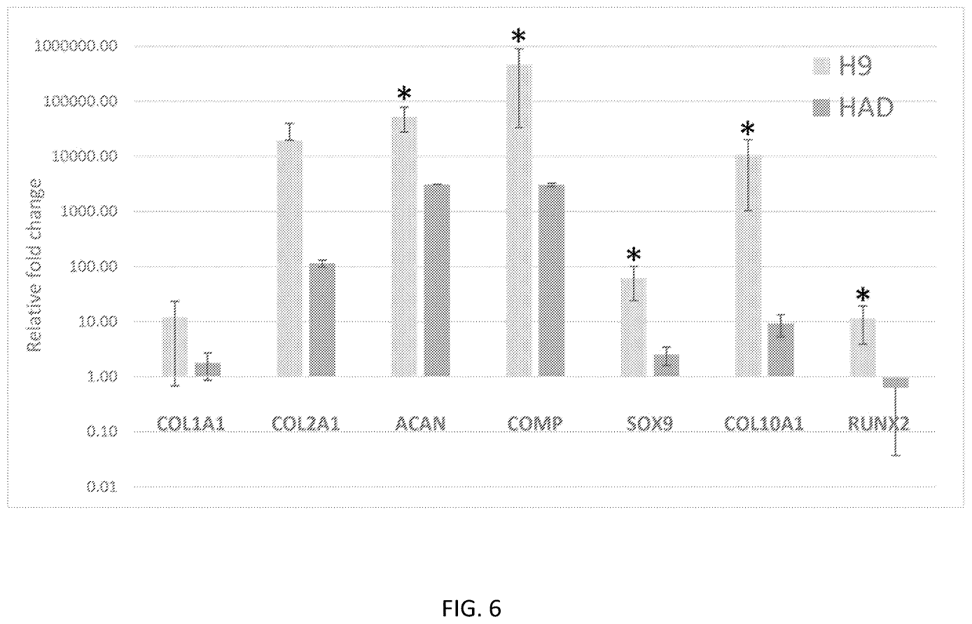

Chondrogenic gene expression was compared between ES-MSC derived from H9 ESC or HADC 100 following exposure to SB431542. Pellets from either ES-MSC source expressed higher levels of chondrogenic genes (COL2A1, ACAN, COMP and SOX9) relative to their respective monolayer controls (FIG. 6). No significant differences were noted between H9 and HAD ES-MSC for COL1A1 and COL2A1 expression. However, significantly higher ACAN (p=0.003), COMP (p=0.04) and SOX9 (p=0.006) was observed in H9 ES-MSC compared to HAD ES-MSC. H9 ES-MSC also expressed higher levels of hypertrophic marker, COL10A1 (P=0.03), and osteogenic transcription factor RUNX2 (p=0.007).

### ES-MSC Embedded in Fibrin-ECM Hydrogels Create Neo-Cartilage Tissues and Integrate in Ex Vivo Osteoarthritic Tissue

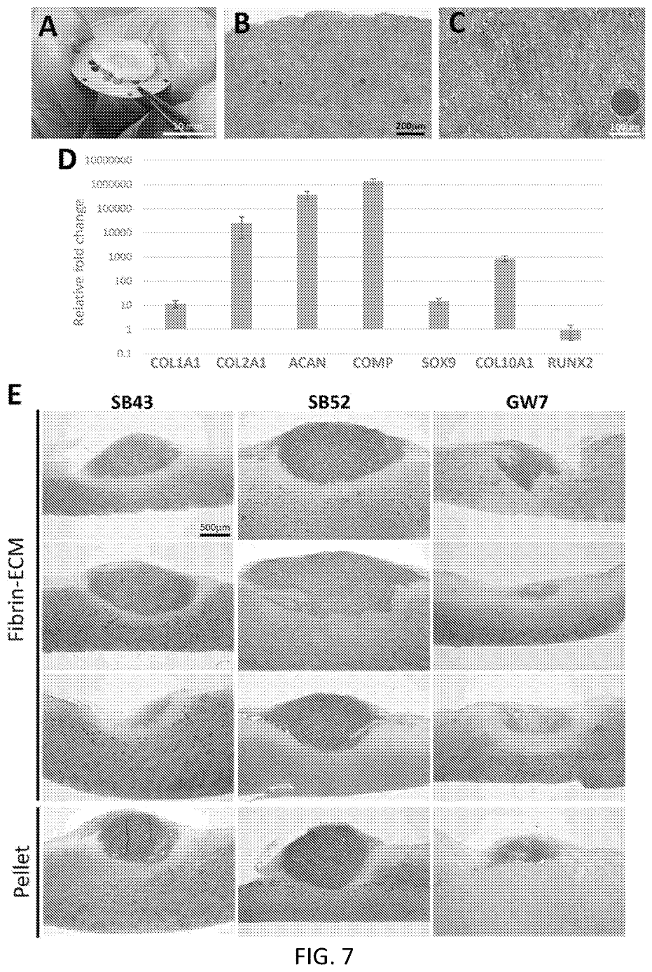

To enable surgical delivery of cells and to facilitate shaping and implantation into chondral or osteochondral lesions, a fibrin hydrogel supplemented with the major ECM components of articular cartilage was developed: collagen type II, hyaluronate, and chondroitin sulfate (fibrin-ECM). HAD ES-MSC differentiated with SB431542 were mixed in the hydrogel at a density of 20×10⁶ cells per ml and mounted upon a several needle arrays (N=8) to maintain size and shape during culture (FIG. 7, panel A). After 3 weeks of culture, a mechanically stable construct was formed on the needle array (FIG. 7, panel A) with production of cartilaginous tissue (Safranin O fast green staining; FIG. 7, panels B and C). The gene expression profile of these neo-tissues was chondrogenic (FIG. 7, panel D) and resembled that of the pellet cultures from the same cell source (FIG. 5). For comparison among ALK5 inhibitors, ES-MSC differentiated with SB431542, SB525334, or GW788388 treatment, and suspended in fibrin-ECM constructs were immediately implanted into defects created in ex vivo osteoarthritic cartilage explants. For comparison, pellet cultures from the same cell sources were also implanted into osteoarthritic explants, in parallel experiments. Following 3-weeks in serum-free chondrogenic medium with TGFβ3 (10 ng/ml), cartilaginous neo-tissues formed equally well in all ALK 5 inhibitor conditions, implanted as hydrogel or pellets (FIG. 7, panel E).

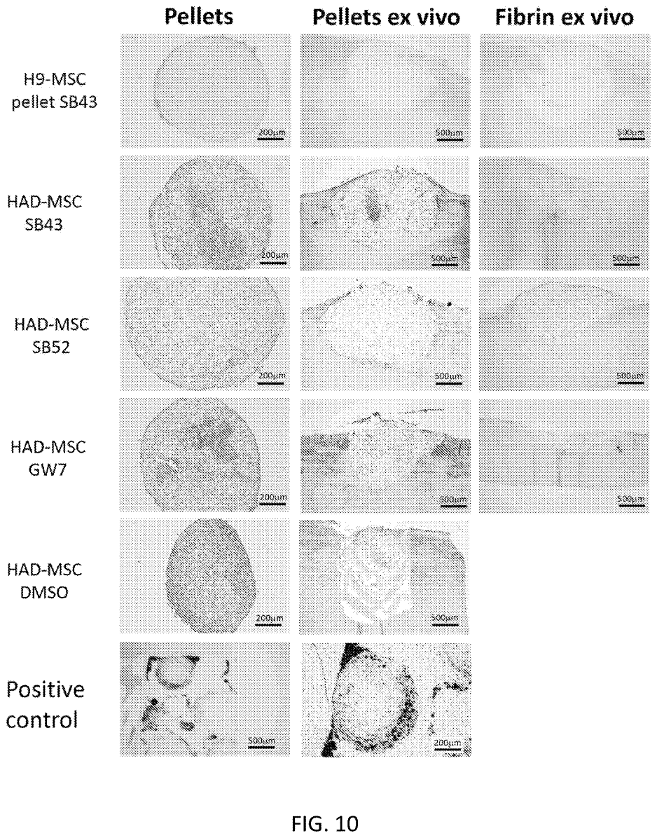

Hypertrophic differentiation is a major concern in adult bone marrow derived MSC and H9-ES-MSC expressed higher levels of COL10A1 and RUNX2 (FIG. 6). Hypertrophic differentiation and mineralization in the neotissues generated from both H9-ES-MSC and HAD-ES-MSC was therefore examined via Alizarin Red S staining. No evidence of mineralization was observed for any cell line studied in pellet culture or in fibrin-ECM hydrogels in free culture or after ex vivo implantation (FIG. 10).

### Discussion

A rapid and simple approach to derive ES-MSC was developed, with high chondrogenic capacity, from the commonly studied H9 ESC cell line and a clinically relevant xeno-free derived HADC100 cell line. Overall, both ESC cell lines responded equivalently to the small molecule differentiation approach used in this study. ALK5 inhibitors (particularly SB525334) successfully induced MSC differentiation in HADC100 cells. ES-MSC from H9 and HADC 100 cells produced high quality neo-cartilage tissues with robust deposition of GAGs and collagen type II, without evidence of mineralization, as pellets in free culture, when encapsulated in fibrin-ECM hydrogel, as well as after implantation into osteoarthritic ex vivo tissue.

An approach to spontaneously derive chondroprogenitors from H9-ESC was previously reported. This approach was relatively simpler than other spontaneous differentiation approaches using Matrigel or gelatin and consecutive enzymatic passaging. However, Olee et al. expanded ESC on mouse embryonal fibroblast feeders and ESC differentiation required relatively longer culture times (up to five passages) for the consistent emergence of an MSC phenotype. In the present study, after expanding H9 ESC without mouse feeders and subsequently culturing on fibronectin coated flasks, chondroprogenitor cells were derived at a lower passage (P1-P2). These results were then reproduced in the xeno-free HADC 100 ESC cell line, initially cultured on human foreskin feeder cells, under otherwise identical differentiation conditions.

ALK-inhibitors have been shown to induce MSC differentiation in various pluripotent lines, under different culture substrates and techniques. Mahmood et al. 2010 suspended HUES9 ESC EBs in 10 μM SB431542 before plating on fibronectin to induce emergence of fibroblastic cells, which were subsequently cultured in monolayer with 10% FBS and continued exposure to SB431542. The resultant cells were highly enriched for MSC markers CD44, CD73, CD146, and CD166; although CD90 and CD105 were not reported. Sánchez et al. 2011 exposed several ESC cell lines (H9, AND-1, AND-2, and SHEF-1) to SB431542 in monolayer cultures on Matrigel coated flasks. This approach led to a fibroblastic cell population that was only 42% CD73+CD90+CD34− (comparable to the results herein with DMSO negative controls); and subsequent sorting for CD73 and CD90 double positive cells was required to increase purity to >85%. Chen et al. 2012 differentiated Mel1 and HES3 ESC lines. The cells cultured in monolayer on Matrigel initially acquired an epithelial-like morphology, eventually differentiating into an MSC-like phenotype upon subsequent culture. Cells cultured as EBs after 10 days of SB431542 treatment and subsequently seeded on standard tissue culture plastic gave rise to a heterogeneous population of cells, with MSC-like surface markers, and the capacity to undergo osteogenesis and chondrogenesis. In general, the method of differentiation disclosed herein was more efficient, did not require xeno-derived reagents (such as Matrigel) and performed equivalently with xeno-free derived HADC 100 ESC.

Although SB431542 is one of the most commonly used ALK5 inhibitor, the efficacy of 2 other ALK5 inhibitors was explored. SB431542 and SB525334 appear to be equally effective in differentiating ES-MSC. SB431542 seemed superior in terms of generating mesoderm markers (such as NCAM1 and SOX9) and increasing the expression of COL2A1 as ES-MSC in monolayer culture. On the other hand, SB525334-treated cells developed neo-cartilage tissues with the most uniform GAGs and collagen type II deposition, and with reduced collagen type I. While these small molecule ALK5 inhibitors are promising, there may be concerns about use of ALK5 inhibitors for clinical applications. However, several ALK5 inhibitors are in clinical trials for various therapies. Therefore, the transient (5-10 days) exposure to cells is less likely to be an issue for tissue engineering cell therapy.

Other small molecules are being explored to produce chondroprogenitors from human pluripotent cells. CHIR99021, a glycogen synthase kinase 3 inhibitor, and TTNPB, a retinoid acid receptor agonist, have been used to produce chondroprogenitors from hiPSC. This simple protocol led to sequential differentiation through mesendoderm, mesoderm, and to chondroprogenitors, leading to abundant collagen type II immunocytochemical staining. However, the pluripotent cells had been cultured on mouse embryonic fibroblast feeders. Bromodomain-containing protein 4 (BRD4) interacts with OCT4 and pluripotency genes, augments histone acetylation, maintains the expression and chromatin patterns of pluripotency-associated genes, and controls the master regulators of mesoderm formation including Brachyury. Combining BRD4 inhibitors (LLY-507 and AZD5153) successfully induced MSC phenotype in pluripotent stem cells after monolayer culture on Matrigel-coated plates with subsequent culture in MSC culture medium.

The feasibility of rapidly deriving ES-MSC and chondroprogenitors with high cartilage forming capacity using the commonly studied H9 ESC cell line and a more clinically relevant xeno-free derived ESC cell line is demonstrated herein. The derived ES-MSC were able to produce high quality neo-cartilage tissues with robust deposition of GAGs and collagen type II, as pellets in free culture, when encapsulated in fibrin-ECM hydrogel, and after implantation into osteoarthritic ex vivo tissue.

### Example 3: ES-MSC Seeded in Microspheriod Building Chamber Create Bone and Cartilage (Osteochondral) Tissues

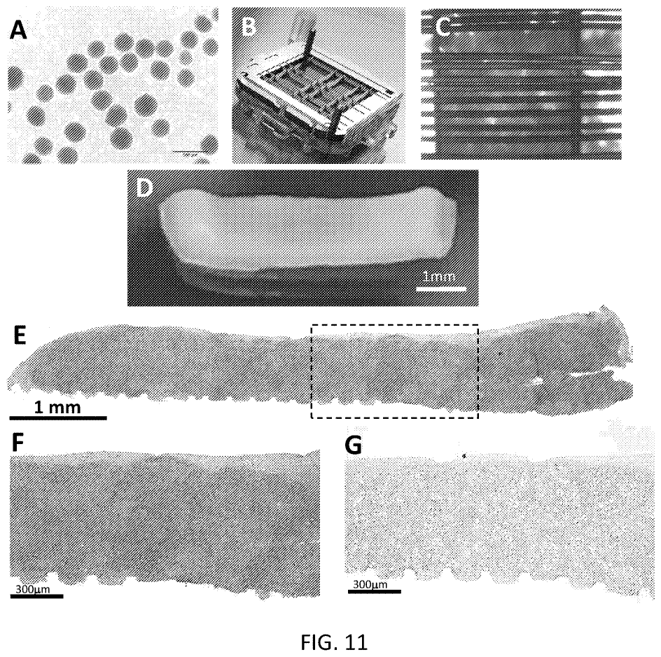

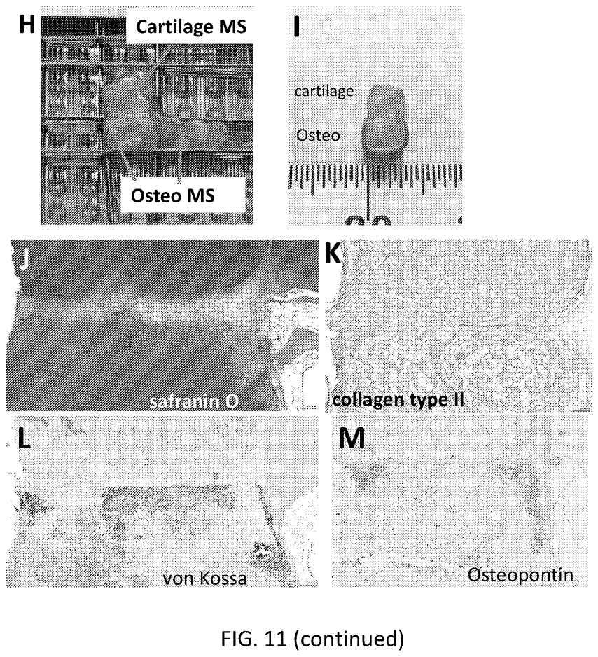

Using a microspheroid building block approach, ES-MSC derived from treatment with ALK-5 inhibitor SB525334, as described in Example 1, were used to produce bone and cartilage (osteochondral) tissues. ES-MSC microspheroids (FIG. 11, panel A) were seeded into a chamber (FIG. 11, panels B and C) for 3 weeks to produce neo-cartilage tissue (FIG. 11, panel D) that was positive for cartilage GAGs (FIG. 11, panels E and F) and positive for collagen type II (FIG. 11, panel G). As shown in FIG. 11, panel H, ES-MSC microspheroids pre-differentiated towards cartilage or towards a calcified cartilage phenotype (Osteo MS) were both seeded into a chamber and cultured in chondrogenic medium to encourage an integrated osteochondral (OC) tissue unit (FIG. 11, panel I). The OC tissue unit was implanted into an ex vivo model to develop biphasic tissue with an upper cartilage and lower calcified cartilage layer (FIG. 11, panels J-M). Both upper and lower tissues were Safranin O and collagen type II positive (FIG. 11, panels J-K), while the lower tissue producing calcified neo tissue (FIG. 11, panel L; von Kossa positive) and osteopontin (FIG. 11, panel M).

### Example 4: ES-MSC Seeded in Blow Spun Collagen Scaffolds Create Meniscus Cartilage-Like Tissues

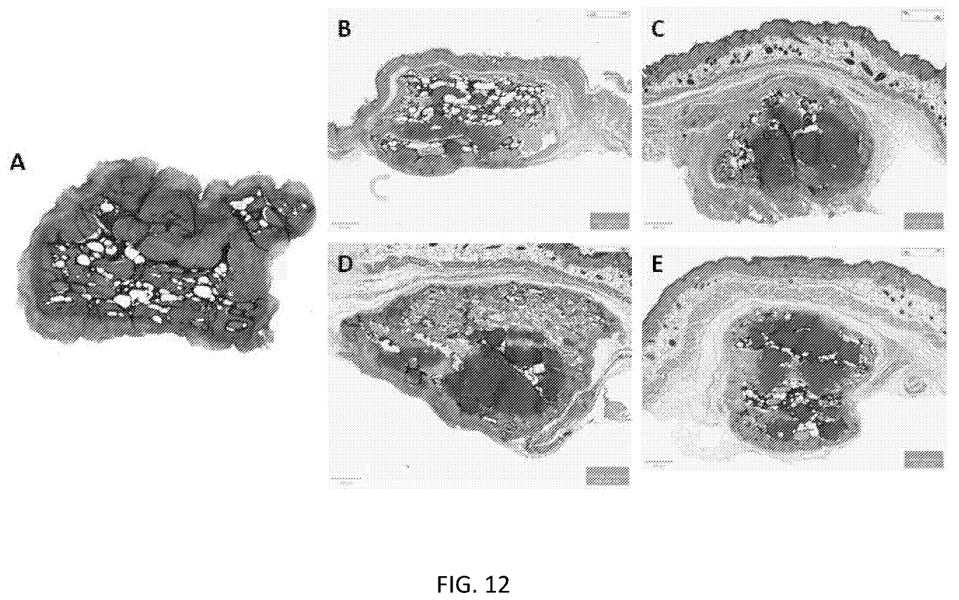

ES-MSC derived from treatment with ALK-5 inhibitor SB525334, as described in Example 1, produced meniscus cartilage-like tissues when seeded upon blow spun collagen scaffolds in in vitro and in vivo. ES-MSC were seeded upon a glutaraldehyde crosslinked pneumatospun collagen type I scaffold and cultured for 6 weeks in chondrogenic medium and stained for Safranin O showing extensive GAG deposition (FIG. 12, panel A). In parallel, scaffolds seeded with ES-MSC and cultured for 1 week in vitro were implanted subcutaneously into a nude mouse for another 5 weeks to produce meniscus cartilage-like tissue with strong Safranin O staining profile (FIG. 12, panels B-E).

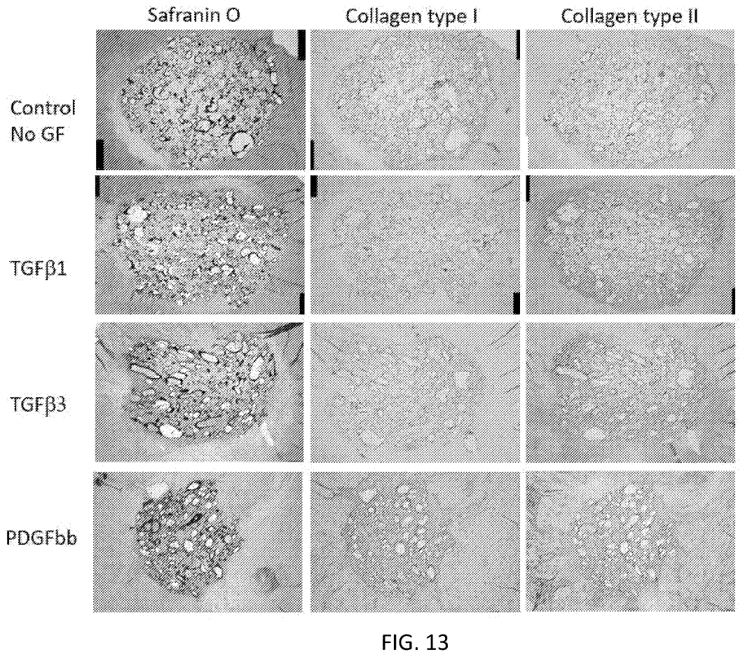

In a similar experiment, meniscus cartilage-like tissues produced by seeding ES-MSC derived from ALK-5 inhibitor SB525334 upon blow spun collagen scaffolds were implanted into ex vivo human meniscus defects. ES-MSC were seeded upon a glutaraldehyde crosslinked pneumatospun collagen type I scaffold (3.5×1 mm disks) that was conjugated with growth factors (TGFb1, TGFb3 or PDGFbb) or unconjugated (control). After 2 weeks in differentiation medium in vitro, the disks were implanted into 3.5 mm defects formed in ex vivo human meniscus tissue and cultured for 5 weeks. As shown in FIG. 13, all growth factor conjugated scaffolds produced Safranin O positive ECM. TGFβ1 and TGFb3 produced neo-meniscus like tissues higher in collagen type II (outer meniscus phenotype), while PDGFbb conjugated scaffolds induced both collagen type I and II rich ECM (FIG. 13).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of generating mesenchymal stem cells (MSCs), comprising:
(a) culturing a population of pluripotent stem cells in xeno-free conditions; and
(b) contacting the population of pluripotent stem cells with an inhibitor of the SMAD2/3 signaling, wherein the population of pluripotent stem cells differentiates into a population of MSCs.

2. The method of claim 1, further comprising, after (a), culturing the population of pluripotent stem cells in suspension spinner culture.

3. The method of claim 1, wherein the population of pluripotent stem cells is xeno-free derived embryonic stem cell (ESCs).

4. The method of claim 1, wherein the population of pluripotent stem cells comprises HADC 100 cell line.

5. The method of claim 1, wherein the MSCs comprise chondroprogenitor cells.

6. The method of claim 1, wherein the inhibitor of the SMAD2/3 signaling is an ALK-5 inhibitor.

7. The method of claim 1, wherein the inhibitor of the SMAD2/3 signaling comprises at least a concentration of 5 μM.

8. The method of claim 6, wherein the ALK-5 inhibitor is SB525334.

9. The method of claim 6, wherein the ALK-5 inhibitor is SB431542.

10. The method of claim 6, wherein the ALK-5 inhibitor is GW788388.

11. The method of claim 1, wherein the population of MSCs is passaged at least two times.

12. The methods of claim 1, wherein the population of MSCs is at least 80% positive for CD73, CD90, CD105, or a combination thereof.

13. The method of claim 1, wherein the population of MSCs is at least 90% positive for CD73.

14. The method of claim 1, wherein the population of MSCs is at least 80% positive for CD90.

15. The method of claim 1, wherein the population of MSCs is at least 75% positive for CD105.

16. The method of any one of claims 1-15, further comprising: (c) passaging and culturing the population of MSCs to form high-density pellets in a medium; and (d) implanting the high-density pellets into ex vivo explants to form a neo-cartilage tissue, a neo-muscular tissue, a neo-meniscus, a neo-tendon, or a neo-bone.

17. The method of claim 16, further comprising culturing the high-density pellets for at least three days before the implanting.

18. The method of claim 16, wherein the medium is serum-free.

19. The method of claim 16, wherein the medium comprises a protein of a TGFβ family.

20. The method of claim 19, wherein the TGFβ family comprises TGFβ1, TGFβ2, or TGFβ3.

21. The method of claim 16, wherein the high-density pellets express one or more markers selected from: COL2A1, ACAN, COMP and SOX9.

22. The method of claim 16, wherein the high-density pellets express significantly less COL10A1 compared to pellets not derived from xeno-free human pluripotent stem cells.

23. The method of claim 16, wherein the high-density pellets express significantly less RUX2A compared to pellets not derived from xeno-free human pluripotent stem cells.

24. The method of claim 16, wherein the neo-cartilage tissue, the neo-muscular tissue, the neo-meniscus, the neo-tendon, or the neo-bone expresses GAGs and collagen type II.

25. The method of any one of claims 1-15, further comprising: (c) adding the population of MSCs to a hydrogel construct; and (d) implanting the hydrogel construct into ex vivo explants to form a neo-cartilage tissue, a neo-muscular tissue, a neo-meniscus, a neo-tendon, or a neo-bone.

26. The method of claim 25, wherein the hydrogel construct is supplemented with extracellular matrix components.

27. The method of claim 26, wherein the extracellular matrix components comprise collagen type II, hyaluronate, chondroitin sulfate, or any combination thereof.

28. The method of claim 16 or 25, wherein the ex vivo explants comprise human osteoarthritic cartilage.

29. The method of claim 16 or 25, wherein the neo-cartilage tissue, the neo-muscular tissue, the neo-meniscus, the neo-tendon, or the neo-bone does not comprise mineralization.

30. The method of claim 16 or 25, wherein the neo-cartilage tissue, the neo-muscular tissue, the neo-meniscus, the neo-tendon, or the neo-bone comprises mineralization.

31. A composition, comprising:
a) a population of pluripotent stem cells in xeno-free culture; and
b) an inhibitor of SMAD2/3 signaling, wherein the inhibitor of SMAD2/3 signaling differentiates the population of pluripotent stem cells to a population of mesenchymal stem cells (MSCs).

32. The composition of claim 31, wherein the population of pluripotent stem cells comprises a HADC 100 cell line.

33. The composition of claim 31, wherein the population of pluripotent stem cells comprise chondroprogenitor cells.

34. The composition of claim 31, wherein the inhibitor of SMAD2/3 signaling is an ALK-5 inhibitor.

35. The composition of claim 31, wherein the inhibitor of SMAD2/3 signaling comprises at least a concentration of at least 5 μM.

36. The composition of claim 34, wherein the ALK-5 inhibitor is SB525334.

37. The composition of claim 34, wherein the ALK-5 inhibitor is SB431542.

38. The composition of claim 34, wherein the ALK-5 inhibitor is GW788388.

39. The composition of claim 31, wherein the population of MSCs are passaged at least two times.

40. The composition of claim 31, wherein the population of MSCs is at least 80% positive for CD73, CD90, and CD105, or a combination thereof.

41. The composition of claim 31, wherein the population of MSCs is at least 90% positive for CD73.

42. The composition of claim 31, wherein the population of MSCs is at least 80% positive for CD90.

43. The composition of claim 31, wherein the population of MSCs is at least 75% positive for CD105.

44. The composition of any one of claims 31-43, wherein the population of MSCs is passaged and cultured to form high-density pellets in a medium.

45. The composition of any one of claims 31-43, wherein the population of MSCs is added to a hydrogel construct.

46. The composition of claim 44, wherein the high-density pellets express one or more markers selected from: COL2A1, ACAN, COMP and SOX9.

47. The composition of claim 44, wherein the high-density pellets express significantly less COL10A1 compared to pellets not derived from xeno-free human pluripotent stem cells.

48. The composition of claim 44, wherein the high-density pellets express significantly less RUNX2 compared to pellets not derived from xeno-free pluripotent stem cells.

49. The composition of claim 44, wherein the high-density pellets are implanted into ex vivo explants to form a neo-cartilage tissue, a neo-muscular tissue, a neo-meniscus, a neo-tendon, or a neo-bone.

50. The composition of claim 45, wherein the hydrogel construct is supplemented with extracellular matrix components.

51. The composition of claim 50, wherein the extracellular matrix components comprise collagen type II, hyaluronate, chondroitin sulfate, or any combination thereof.

52. The composition of claim 45, wherein the hydrogel construct is implanted into ex vivo explants to form a neo-cartilage tissue, a neo-muscular tissue, a neo-meniscus, a neo-tendon, or a neo-bone.

53. The composition of claim 49 or 52, wherein the ex vivo explants comprise human osteoarthritic cartilage.

54. The composition of claim 49 or 52, wherein the neo-cartilage tissue, the neo-muscular tissue, the neo-meniscus, the neo-tendon, or the neo-bone does not comprise mineralization.

55. The composition of claim 50 or 53, wherein the neo-cartilage tissue, the neo-muscular tissue, the neo-meniscus, the neo-tendon, or the neo-bone comprises mineralization.

56. A population of cells, wherein the population is
(a) at least 90% positive for CD73;
(b) at least 80% positive for CD90; and
(c) at least 75% positive for CD105;
wherein the population of cells has a reduced expression of one or more hypertrophic markers.
